(19) United States
(12) Patent Application Publication
(10) Pub. No.: US 2022/0310261 A1
(43) Pub. Date: Sep. 29, 2022

(54) CLINICAL DECISION SUPPORT SYSTEM FOR ESTIMATING DRUG-RELATED TREATMENT OPTIMIZATION CONCERNING INFLAMMATORY DISEASES

(71) Applicant: Siemens Healthcare GmbH, Erlangen (DE)

(72) Inventors: Asmir VODENCAREVIC, Fuerth (DE); Melanie HANKE, Fuerth (DE); Jan JAKUBCIK, Zilina (SK); Volker SCHALLER, Uttenreuth (DE); Andre WICHMANN, Buckenhof (DE); Peter ZIGO, Zilina (SK); Marcus ZIMMERMANN-RITTEREISER, Grossenseebach (DE)

(73) Assignee: Siemens Healthcare GmbH, Erlangen (DE)

(21) Appl. No.: 17/703,226

(22) Filed: Mar. 24, 2022

(30) Foreign Application Priority Data
Mar. 29, 2021 (EP) 21165619.4

(51) Int. Cl. G16H 50/20; G16H 20/10

(52) U.S. Cl. CPC G16H 50/20; G16H 20/10

(57) ABSTRACT

A clinical decision support system for estimating drug-related treatment optimization concerning inflammatory diseases, comprises: a computing unit configured to host a plurality of prediction models, the computing unit including an input interface designed for receiving input data and an output interface designed to output result; a plurality of different trained prediction models, each model trained to predict the probability of treatment outcomes for a number of different drug-related treatment options and for a specific patient-group; a selection unit configured to automatically select one a prediction model depending on the input data according to a predefined selection scheme. The clinical decision support system is configured to produce output results by processing the input data with the selected prediction model.

### CROSS-REFERENCE TO RELATED APPLICATION(S)

The present application hereby claims priority under 35 U.S.C. § 119 to European patent application no. EP 21165619.4, filed Mar. 29, 2021, the entire contents of which are hereby incorporated herein by reference.

### FIELD

Embodiments of the present invention describe a clinical decision support (“CDS”) system for estimating drug-related treatment optimization concerning inflammatory diseases, as well as a prediction-method of computed decision support and a method for manufacturing such CDS system.

### BACKGROUND

Inflammatory diseases, such as rheumatic diseases like rheumatoid arthritis, psoriasis arthritis, other musculoskeletal diseases, Chronic Obstructive Pulmonary Disease (COPD), asthma, multiple sclerosis or Crohn's disease, include a wide range of medical conditions, causing chronic pain and inflammation. For example, rheumatic diseases affect joints, tendons, ligaments, muscles and bones. The most of these conditions occur when the immune system starts attacking its own tissue for still unclear reasons. Often inflammatory diseases are characterized by interlaced periods of disease inactivity, also called “remission”, low and moderate disease activity as well as periods of exacerbated (high) disease activity, known as “flares”.

While the most of such diseases cannot be cured, there are different types of medications which can help to keep the disease activity at low levels. Appropriate medication and dosage are e.g. specified in disease-specific guidelines (such as the ACR and the EULAR guidelines for rheumatoid arthritis). However, they are derived based on clinical studies and statistical analysis on cohort level.

Due to large differences in patients' characteristics such as demographics, diet, and lifestyle, genetic predispositions, susceptibility to external factors such as weather conditions, and likely other factors as well, it remains challenging for rheumatologists to find the right medication and/or the right dosage for an individual patient.

Often, an effective treatment needs to be changed to accommodate patient's current situation (e.g. pausing immunosuppressive therapy due to planned surgery or acute infections), lower the risk of adverse events of medication and/or reduce healthcare costs.

Furthermore, according to the “treat-to-target” strategy described in the guidelines for treating rheumatoid arthritis, the dosage of drugs and especially biologic drugs should be tapered once the stable remission is achieved. The rheumatologist is then again faced with the challenge, which patients are eligible for tapering and how much can the drug be tapered in each individual case. This is often a trial-and-error process, accompanied by reduced patient quality of life and increased healthcare costs until the correct treatment is found.

In the ambulatory clinical routine of rheumatic patients, they are examined in regular or irregular (e.g. in the case of complications like flares) time intervals by rheumatologists. During a typical patient visit, examination data is collected and sometimes previously collected demographic and lifestyle data is confirmed or updated. Based on this data, the rheumatologist makes a treatment decision ideally together with the patient. During patient's visit, a blood sample is typically taken and sent to a laboratory for analysis, most often focusing on inflammation biomarkers, such as C-reactive protein (CRP). The results of this blood test become available later, normally several days after patient's visit and after the treatment decision has already been made.

In the light of newly available lab data, the rheumatologist sometimes gains new insights and adjusts patient's treatment per phone. In all cases, treatment decisions are based on multiple relevant variables relating to patient's demographics (e.g. age, gender), examination data (e.g. patient questionnaires, numbers of tender and swollen joints), blood values (e.g. ESR, CRP), and medications (e.g. substance, co-therapy, dosage etc.).

Finally, clinical guidelines emphasize shared decision making between clinicians and patients regarding the treatment, which is not an easy task to accomplish given high number of involved relevant variables.

Specific problems are:

1. Physicians (e.g. rheumatologists) often struggle to find the initial medication and/or dosage which is likely to work for an individual patient.
2. Physicians often need to taper the drug dosage, not knowing if and how much tapering is safe and still effective for an individual patient.
3. Data relevant for treatment decisions becomes available at different time points.
4. Due to the complex nature of high-dimensional decision making in the field of inflammatory diseases (e.g. in rheumatology), data-driven black-box decision support systems are often conceived lacking transparency, which negatively affects their acceptance both by clinicians and patients.

### SUMMARY

So it is an object of embodiments of the present invention to improve the known methods and provide a clinical decision support system for estimating drug-related treatment optimization concerning inflammatory diseases, especially a data-driven clinical decision support for therapy planning in rheumatic disease.

An object of embodiments of the present invention is achieved by the clinical decision support system, a prediction-method, a method for manufacturing a CDS system and a data processing system.

In the following, embodiments of the present invention may be described using examples with respect to predicting the probability of flares of rheumatoid arthritis, but the present invention is not limited to this application. Embodiments of the present invention and its aspects can be used in particular for predicting the future status of a patient having a known inflammatory disease, like e.g. psoriasis arthritis, other musculoskeletal diseases, Chronic Obstructive Pulmonary Disease (COPD), asthma, multiple sclerosis or Crohn's disease.

A clinical decision support system according to embodiments of the present invention for estimating drug-related treatment optimization concerning inflammatory diseases, comprises the following components:
a computing unit designed to host a plurality of prediction models, the computing unit comprising an input interface designed for receiving input data and an output interface designed to output results,—a plurality of different trained prediction models, wherein each model is trained to predict the probability of treatment outcomes for a number of different drug-related treatment options and for a specific patient-group based on input data,
a selection unit designed for automatically selecting one of these prediction models depending on the input data according to a predefined selection scheme, wherein the CDS system is designed to produce output results by processing the input data with the selected prediction model.

In general, clinical decision support (CDS) systems are known in the art. However, this CDS system provides an estimation of a drug-related treatment optimization risk probability within a future time period. The expression “drug-related” in this context may mean in relation to a drug response and/or side effects concerning drug dosage and/or type of drug. Alternatively, “drug-related” may mean the (negative or positive) reaction of a patient on an applied drug (of a certain dose). For example, for a certain amount or type of a drug the drug response probability may be estimated (whether a drug helps or not) and/or the risk of side effects for a certain patient may be predicted.

A suitable computing unit should have enough memory and computing power to host the plurality of prediction models. This means that the computing unit must be able to process these prediction models in order to get results from input data from the prediction models. However, prediction models not used do not necessarily need to be hosted by the computing unit. They could e.g. be present in a memory until needed. Such computing units with an input interface and an output interface are known in the art. The output interface may be a data interface or a display. Any manner, apparatus or device for outputting results are possible as long as they are able to provide the data format desired by the user.

Concerning the prediction models, these are models trained for different purposes. Preferably they have been trained with training data of different groups of patients (each of these models with a training dataset concerning a different group of patients) and/or with training data relating to different medications (although a model may also be trained with a group of different drugs). The models may also be trained with different types of data (e.g. demographic data, medication data, examination data, or lab data), e.g. one model is trained with laboratory data and one with demographic data. In general, the training of prediction models as well as the architecture of these prediction models are well known in the art (see e.g. EP 3 573 068 A1).

The prediction models all have in common that each model is trained to predict the probability of treatment outcomes based on input data. These treatment outcomes may concern the response of a patient to a drug in a positive way (relief of pain, improvement of the condition), a negative way (side effects) or a neutral way (no response at all). Thus, “treatment outcomes” may be read as side effects and/or disease outcomes. This is done for a number (one or more) of different drug-related treatment options, e.g. different doses of a certain group of drugs (or a number of such groups) or different drugs. Additionally, this is done for a specific patient-group. Preferably, there are in total more than 10 different trained models used, especially more than 30 or even more than 50.

The models may be present in the computing unit itself or in a memory used by the computing unit. For example, information about the architecture and parameters of the prediction models are present in a memory and a chosen prediction model is downloaded from the memory into (a random access memory of) the computing unit.

The selection unit is designed for automatically selecting one of these prediction models. The selection is based on a predefined selection scheme and on data inputted in the CDS system to be processed by the models. The selection scheme may be a table stored in a memory of the computing system or a decision tree hardwired in the algorithm of the selection unit. Depending on the inputted data (e.g. diagnosis, lab data, data comprising information about certain drugs applied to a patient, age or gender of the patient), a model of the plurality of models is selected by the selection unit based on the selection scheme. Such selection is beneficial because it is very complicated (or even impossible) to train one single prediction model for all possible cases and for all possible patients. Furthermore, sometimes it turns out that for a special case (a special group of patients, a special disease or a special use case), a prediction model of a different architecture is better than a model with another architecture. Thus, embodiments of the present invention can evaluate, which prediction model (architecture and training) would be optimal for what case while constructing the CDS system and this prediction model is chosen to be part of the CDS system. Then, if the special case occurs, this model is selected by the selection unit and provides the best results for the special case.

The selection unit is preferably designed to search the input data for predefined data types and/or for values of predefined data types and to determine whether a predefined data type is present in the input data and/or to compare a value with a predefined threshold and/or to decide if the value fits a predefined requirement. For example, the selection unit may be designed to look, whether the gender of a patient is male, female or undefined, or to look whether there is lab-data available in the input data, or if diagnosis is rheumatoid arthritis for example.

The CDS system is designed to produce output results by processing the input data with the selected prediction model. How to process data with a trained model is known in the art.

Thus, depending on the used models and the selection scheme, the CDS system is able to predict response of a patient to a certain drug (concerning side effects and/or ease), or predict worsening during therapy de-escalation (especially “flare” prediction in PsA and RA and “exacerbation” prediction in COPD and Asthma). Regarding COPD, there is e.g. no de-escalation of biologics since they are to date not approved for treating these diseases but there is de-escalation of other applicable drugs, especially corticosteroids.

For example, if a patient suffering from rheumatoid arthritis is entering phase 1 of a treatment, the CDS system can estimate, whether this patient will positively respond to drugs that are suggested by corresponding guidelines, approved for use and available at the treating institution. If the model will predict that this special patient will not respond to any phase 1 drugs, then phase 2 could be started immediately, sparing months of suffering due to non-effective drugs or side effects.

A prediction-method according to embodiments of the present invention of computed decision support comprises the following steps:
providing a CDS system according to embodiments of the present invention,
providing input data to the CDS system, wherein the input data is preferably selected and provided automatically, especially if new data becomes available for a predefined patient,
determining a result with the CDS system wherein a prediction model is selected automatically by the CDS system based on the input data and the result is determined automatically by the selected prediction model,
outputting the result, especially wherein a user is notified if substantial changes in a result for a patient occurred compared to earlier results for the same patient, e.g., in the form of a warning message or an icon in the patient list, so that he knows that he has to open the case.

The input data is preferably data relating to one single patient and preferably comprises data from the group of demographic data (possibly including lifestyle data), medication data, examination data and lab data. Additionally, the input data comprises information about an intended change of treatment, e.g. information about a drug intended to be applied to the patient or a reduction or increase of a drug dose. Moreover, the input data could potentially include omics data (e.g. proteomics, genomics, metabolomics) and medical image data acquired using different imaging modalities (e.g. magnetic resonance imaging, ultrasound, computed tomography).

A method according to embodiments of the present invention for manufacturing a CDS system according to embodiments of the present invention comprises the following steps:
providing at least a first model-group and a second model-group, each model-group having a plurality of untrained machine learning models, especially with a number of models having a different internal architecture and/or different hyperparameters,
providing at least a first training-dataset and a second training-dataset, each training-dataset comprising data with a different distinguishing feature,
training of the first model-group with the first training-dataset and the second model-group with the second training-dataset,
ranking each trained prediction model of a model-group with a predefined quality-criterion, preferably wherein prediction models are developed and compared offline,
choosing the best ranked prediction model of each model-group as prediction model for the clinical decision support system manually or automatically.

It should be noted that the model-groups mentioned here are not the models of the CDS system. Only the “winner” of a group (or winners) will take a place in the final CDS system. In praxis, there should be more groups than one, e.g. more than 10, more than 30 or more than 50.

The training datasets should be chosen on behalf of the purpose of the individual trained model. If a model is to be applied for patients of the age of 60 or older, the training dataset should only comprise data of patients of the age 60 or older. If the model is to be applied for a certain drug, the respective training dataset should comprise data about patient response based on this drug.

The training criterion could depend on the state of the patients the training data were taken from. The criterion could allocate a quality score to a prediction model in the case a validation occurs and the prediction quality of the trained models is quantized. The training criterion could also be derived from the criterions of a nested cross validation process.

For example, there is a number of predictive models (e.g. 52) trained for different diseases, medications, actions (application of new drugs or drug-reduction) and patient-groups. These models could then be used in the CDS system for, e.g. RA (rheumatoid arthritis); Phase II (EULAR guidelines); prediction of response to a certain drug, or PsA (psoriasis arthritis); Phase IV, tapering (also known as “dose reduction” or “therapy de-escalation”). The best model is automatically chosen for the given purpose by the selection unit.

Regarding a patient with a certain disease, the selection can be accomplished by determining which of the predictive models is trained with a group of patients that has the most similarities with the actual patient, respectively which of the predictive models is trained with patients having the actual disease. Regarding information about a (planned or applied) medication in the input data, it may be determined, which of the predictive models is trained with a group of patients getting these drugs. Regarding certain types of input data (e.g. lab data), it may be determined, which of the predictive models is trained with such data.

A data processing system of embodiments of the present invention, that is especially a computer network system, comprises a data-network, a number of client computers and a service computer-system, wherein the service computer system comprises a Clinical Decision Support System according to embodiments of the present invention.

The units or modules of embodiments of the present invention mentioned above can be completely or partially realised as software modules running on a processor of a computing system. A realisation largely in the form of software modules can have the advantage that applications already installed on an existing system can be updated, with relatively little effort, to install and run the methods of the present application. The object of embodiments of the present invention is also achieved by a computer program product with a computer program that is directly loadable into the memory of a computing system and which comprises program units to perform the steps of the inventive method when the program is executed by the computing system. In addition to the computer program, such a computer program product can also comprise further parts such as documentation and/or additional components, also hardware components such as a hardware key (dongle etc.) to facilitate access to the software.

A computer readable medium such as a memory stick, a hard-disk or other transportable or permanently-installed carrier can serve to transport and/or to store the executable parts of the computer program product so that these can be read from a processor unit of a computing system. A processor unit can comprise one or more microprocessors or their equivalents.

Particularly advantageous embodiments and features of embodiments of the present invention are given by the following description. Features of different claim categories may be combined as appropriate to give further embodiments not described herein.

Regarding the trained prediction models or their training, there are some parameters (or “data”) that refer to different types of data. There is preferably demographic data, medication data, examination data or laboratory data (also including related scores and derived variables).

Demographic data may be data referring to patient, especially gender (male, female), height, weight, body mass index, age, smoking status (never smoked, yes, ex), alcohol intake (yes, no, amount), list of comorbidities, time since a diagnosis has been made.

Preferred medication data is data referring to a treatment with an active agent, preferably as listed below, especially biologics/biosimilars, methotrexate, other conventional disease-modifying antirheumatic drugs (cDMARDs), targeted synthetic disease-modifying antirheumatic drugs (tsDMARTs), non-steroidal anti-inflammatory drugs (NSAID), glucocorticoids. Referring to any of the active agents, the data may refer to any member of the group treatment (yes/no), actual substance, administration way, prescribed dosage, prescribed interval, start time and stop time.

Preferred examination data is data referring to a tender joint count (e.g. 0 to 28), swollen joint count (e.g. 0 to 28), patient assessment of pain (e.g. 0 to 100), patient assessment of disease activity (e.g. 0 to 100), doctor assessment of disease activity (e.g. 0 to 100), health Assessment Questionnaire (e.g. 0 to 3), “Funktionsfragebogen Hannover” (e.g. 0 to 100), clinical disease activity index (e.g. 0 to 76).

Preferred lab data is data referring to the rheumatoid factor (positive, negative), Anti-Cyclic Citrullinated Peptides (positive, negative), seropositive rheumatoid arthritis (positive, negative), C-Reactive protein (e.g. >0.01), erythrocyte sedimentation rate (e.g. 0 to 100), Disease Activity Score based on ESR (e.g. 0 to 9.1), Disease Activity Score based on CRP (e.g. 0 to 8), simple disease activity index (e.g. 0 to 86), duration of remission (e.g. >=0), count of previous flares (e.g. >=0).

All these possible data could be included in the input data and be used by the selection unit.

According to an embodiment of the CDS system, for a number of the different prediction models, each prediction model has been trained for a different patient-group and is selected based on patient-relating information in the input data, preferably based on demographic data and/or on examination data, especially based on information concerning distinguishing features from the group comprising gender, type of disease (e.g. seropositive vs. seronegative), age, underlying health condition, body mass index. This means that there are prediction models that are specially trained for special groups of patients that can be recognized by special values of patient related data. For different patients with different respective values, different prediction models are automatically chosen.

According to an embodiment of the CDS system, for a number of the different prediction models, each prediction model has been trained for a different location in a clinical pathway and is selected based on input data referring to the patient's location in a clinical pathway, preferably based on examination data.

According to an embodiment of the CDS system, for a number of the different prediction models, each prediction model has been trained for a different medication and is selected based on a type of medication given (indicated) in the input data, the medication especially based on cDMARDs (lightweight cheaper conventional disease modifying antirheumatic drugs), e.g. on methotrexate, sulfasalazine, hydroxychloroquine and leflunomide. There are however many biologic DMARDs (expensive, partially severe side effects but typically much higher effect on the disease activity). More recently, there are also biosimilars and targeted synthetic DMARDs on the market. And there are also NSAID (e.g. aspirin, ibuprofen) for very light symptoms and also dangerous steroidal drugs like glucocorticoids or cortisone for short-term application in cases of acute severe flares.

Suitable active agents (medication) for special inflammatory diseases are listed below:

Preferred drugs used in the treatment of rheumatoid arthritis are conventional disease-modifying antirheumatic drugs (cDMARD) or biologics or other drugs that temporarily ease pain and inflammation. Preferred cDMARDs used to treat RA include hydroxychloroquine, leflunomide, methotrexate, sulfasalazine or minocycline. Preferred biologics include abatacept, rituximab, tocilizumab, anakinra, adalimumab, etanercept, infliximab, certolizumab pegol or golimumab. Preferred tsDMARDs include Janus associated kinase inhibitors like tofacitinib or baricitinib. Preferred nonsteroidal anti-inflammatory drugs (NSAIDs) comprise ibuprofen/hydrocodone, ibuprofen/oxycodone, naproxen sodium, aspirin, celecoxib, nabumetone, naproxen (-sodium), piroxicam, diclofenac, diflunisal, indomethacin, ketoprofen, etodolac, fenoprofen, flurbiprofen, ketorolac, meclofenamate, mefenamic acid, meloxicam, oxaprozin, sulindac, salsalate, tolmetin, diclofenac/misoprostol, topical capsaicin or opioid pain drugs like codeine, acetaminophen/codeine, fentanyl, hydrocodone, hydromorphone, morphine, meperidine, oxycodone, tramadol. Preferred steroidal drags include corticosteroids like betamethasone, prednisone, dexamethasone, cortisone, hydrocortisone, methylprednisolone, prednisolone. Preferred immunosuppressants comprise cyclosporine, cyclophosphamide, azathioprine or hydroxychloroquine.

Preferred drugs used in the treatment of psoriatic arthritis (PsA) include disease-modifying anti-rheumatic drugs (DMARD) immunosuppressants, and tumor necrosis factor-alpha (TNF-alpha) inhibitors. Preferred DMARDs used to treat PsA include methotrexate, sulfasalazine, cyclosporine or leflunomide. Preferred nonsteroidal anti-inflammatory drugs (NSAIDs) comprise ibuprofen or naproxen. A preferred immunosuppressant drug comprises azathioprine. Preferred TNF-alpha inhibitors comprise adalimumab, etanercept, golimumab or infliximab.

Preferred drugs used in the treatment of Chronic Obstructive Pulmonary Disease (COPD) are for example short-acting bronchodilators, corticosteroids, methylxanthines, long-acting bronchodilators, combination drugs, roflumilast, mucoactive drugs, vaccines, antibiotics, cancer medications or biologic drugs. Examples of short-acting bronchodilators include albuterol, levalbuterol, ipratropium or albuterol/ipratropium. Preferred corticosteroids include fluticasone or prednisolone. Preferred long-acting bronchodilators are aclidinium, arformoterol, formoterol, glycopyrrolate, indacaterol, olodaterol, revefenacin, salmeterol, tiotropium or umeclidinium. Recommended LABA/LAMA combination bronchodilator therapies include aclidinium/formoterol, glycopyrrolate/formoterol, tiotropium/olodaterol or umeclidinium/vilanterol. Combinations of an inhaled corticosteroid and a long-acting bronchodilator include budesonide/formoterol, fluticasone/salmeterol or fluticasone/vilanterol.

Preferred drugs used in the treatment of asthma are bronchodilators or anti-inflammatories, respectively quick-relief medications or long-term asthma control medications. Preferred are short-acting beta agonists like albuterol or levalbuterol. Preferred are also anticholinergic like ipratropium bromide (Atrovent HFA). Preferred long-term asthma control medications comprise inhalable corticosteroids like beclomethasone, budesonide, flunisolide, fluticasone or mometasone; corticosteroids like prednisone, methylprednisolone or hydrocortisone; long-acting beta agonists like formoterol or salmeterol. Preferred combination inhalers comprise budesonide and formoterol or fluticasone and salmeterol. Preferred leukotriene modifiers comprise montelukast, zafirlukast or zileuton. Preferred methylxanthines comprise theophylline. Preferred immunomodulators comprise mepolizumab, omalizumab or reslizumab.

Preferred drugs used in the treatment of multiple sclerosis (MS) are interferon beta-1b, interferon beta-la, glatiramer acetate, peginterferon beta 1-a, mitoxantrone, natalizumab, fingolimod, or other sphingosine-1-phosphate receptor modulators, teriflunomide, pyrimidine, cladribine, ocrelizumab, siponimod, cladribine, diroximel fumarate, ozanimod, monomethyl fumarate.

Preferred drugs used in the treatment of Crohn's disease are medications to treat any infection (normally antibiotics) and to reduce inflammation (normally aminosalicylate anti-inflammatory drugs and corticosteroids). Medications used to treat the symptoms of Crohn's disease especially include 5-aminosalicylic acid (5-ASA) formulations, prednisone, immunomodulators such as azathioprine (given as the prodrug for 6-mercaptopurine), methotrexate, infliximab, adalimumab, certolizumab, vedolizumab, ustekinumab and natalizumab. Hydrocortisone should be used in severe attacks of Crohn's disease.

Some of the above mentioned active agents, e.g. the TNF-alpha inhibitors or immunomodulators belong to the group of expensive biologic drugs.

A preferred embodiment of the CDS system is designed to select a prediction model based on the types of input data available, preferably wherein a prediction model is selected depending on the case whether or not lab data is part of the input data. This has the advantage that in the case of a preliminary talk or examination (without lab results) a prediction model can be chosen that allows a first impression of possible results and after lab data is available, another prediction model is automatically chosen that allows an enhanced and optimized prediction. The availability of distinct data items may be independent from the pathway (also for a non-treatment-naïve patient there may be no recent lab data available). A selection based on the different kinds of data available is advantageous. If lab data is not available other models should be used than with lab data available.

Since there are many possible constellations of input data and inflammatory diseases, in the following there are listed some explaining examples referring rheumatoid arthritis.

There are three phases listed in the EULAR guidelines for treatment of RA with pharmacological non-topical treatments, cDMARDs, biological disease-modifying antirheumatic drugs (bDMARDs) and targeted synthetic disease-modifying antirheumatic drugs (tsDMARDs).

In the first phase, there is often made a selection between methotrexate and sulfasalazine combined with short term glucocorticoids. The CDS system could be used to predict the effectiveness of these drugs and provide help for the estimation of the applied drug. The input data for the prediction could be demographic data and examination data of the respective patient. Predictive models could be trained on said drugs and the response of patients concerning these drugs.

However, in this phase also a predictive model could be selected if a tapering of an already applied drug is planned (i.e. if patient is in sustained remission). Then, the input data for the prediction could comprise demographic data and examination data of the respective patient together with information about the applied drug. Predictive models could be trained on dose reduction in sustained remission scenarios of the respective drug and the response of patients to tapering.

In the second phase, expensive active agents are typically applied. Often, a selection is made between the addition of a bDMARD or a JAK-inhibitor on the one hand and the change of the already applied cDMARD or an addition of a cDMARD on the other hand. The CDS system could be used to predict the effectiveness of these alternatives and provide help for the selection. The input data for the prediction could again be demographic data and examination data of the respective patient in addition to data about the applied drugs. Predictive models could be trained on said data and the response of patients concerning the respective drugs.

However, in this phase also a predictive model could be selected in the case dose reduction or an interval increase is planned in sustained remission. Then also input data for the prediction could be demographic data and examination data of the respective patient together with information about the applied drugs. Predictive models could be trained on dose reduction or interval increase in sustained remission scenarios of the respective drugs and the response of patients.

In the third phase, there is often made a decision whether an applied medication should be changed (e.g. another bDMARD or a JAK-inhibitor). The CDS system could be used to predict the effectiveness of such change. The input data for the prediction could be demographic data and examination data of the respective patient in addition to the applied drugs. Predictive models could be trained on said drugs and the response of patients concerning these drugs.

However, as in phase 2, in this phase also a predictive model could be selected in the case dose reduction or an interval increase is planned in sustained remission. Then also input data for the prediction could be demographic data and examination data of the respective patient together with information about the applied drugs. Predictive models could be trained on dose reduction or interval increase in sustained remission scenarios of the respective drugs and the response of patients.

For example, one study showed that in phase 1, methotrexate is not effective with about 43% of the patients (see e.g. https://arthritis-research.biomedcentral.com/articles/10.1186/s13075-018-1645-5). Thus, it would be advantageous to identify those patients that positively respond to methotrexate and those who do not. In the course of dose reduction or interval increase, the probability of flares (within a certain time horizon) could be computed by the prediction models.

Concerning psoriasis arthritis, there is a similar EULAR guideline comprising four phases with a similar procedure as described above. Here also, effects of the application of a new drug or the risk of flares following drug-tapering could be predicted by automatically selecting a predictive model.

Preferably, a first selection of a predictive model is based on the diagnosis of a physician (type of disease and phase of EULAR guideline), and the demographic data of the patient. Then the drug(s) applied or planned to apply could be part of the input data, as well as the planned actions (response prediction or dose reduction). Last the presence of certain data types (e.g. only examination data or also lab data) may also be a criterion of selection of the used prediction model. Last, historic data (anamnesis of the patient), co-morbidities or lifestyle of a patient, potentially available omics data (e.g. metabolomics, proteomics, genomics) and imaging data (e.g. computed tomography images) may also be part of the input data and basis for selection of a predictive model.

According to an embodiment of the CDS system, a number of the different prediction models is trained to determine a probability that an individual patient will respond to a specific drug and/or a risk of flares for different drug tapering scenarios and/or a risk of drug adverse events.

It is preferred that a prediction model is trained for
determining a response probability for first line drugs, e.g. methotrexate and sulfasalazine, and/or
determining a selection of the second line drug and/or
drug tapering in any treatment stage (according to EULAR tapering recommendations), especially for RA patients receiving biologics in stable remission, preferably for a plurality of dosage regimes.

According to an embodiment of the CDS system, a number of prediction models is trained to determine drug response of a patient for a plurality of drugs, preferably wherein one single model determines drug response of a patient for a plurality of drugs, and/or a model of a group of multiple models determines drug response of a patient for one single drug.

A preferred embodiment of the CDS system is designed to output a probability of a flare (especially connected to the application and/or a dosage of a predefined medication), a probability of an adverse events (e.g. side effects of medication) and/or a probability of a patient non responding to a drug. For example, the CDS system is designed to predict the numeric value of relevant disease activity scores such as DAS28-ESR in RA patients (e.g. instead or additionally to predicting flares), which are defined as DAS28-ESR>2.6.

Preferably prediction models of the clinical decision support system are trained to determine and output a confidence score for a prediction, preferably wherein a prediction is a binary value referring to a classification and the confidence is a probability value and/or preferably wherein the prediction is a regression the output comprises prediction intervals for point predictions.

A preferred embodiment of the CDS system is designed to output information about which input group of parameters affect the output the most, preferably designed to generate for individual parameters of this group a value of how much they affect the output information. This provides a quantitative impression of the importance of these parameters. For example, if it is evident, that for a certain patient a result strongly depends on the body mass index, there could be made specific efforts to positively change the body mass index. Thus, an advantage of this embodiment is that a user could infer from the output that a high flare risk is due to a specific medication regime and selectively change it.

According to an embodiment of a method for manufacturing a CDS system, a prediction method according to embodiments of the present invention is performed with the clinical decision support system according to embodiments of the present invention and a feedback-dataset is provided for a number of patients, wherein the prediction models are further trained with this feedback dataset. It should be noted that the prediction models are connected to the distinguishing feature of the feedback data. Preferably, a feedback-dataset is used for training, where a patient had a flare with a DAS28_ESR score higher than 2.6.

In the preferred case of a CDS system for rheumatoid arthritis (but also applicable for other diseases), there is a plurality of prediction models (especially more than 10), that are specially trained for different input data.

There could be several groups of such prediction model, wherein each group comprises a plurality of prediction models (especially more than 10), that are specially trained for different input data for different diseases (e.g. RA, PsA, Spondyloarthropathy—SpA). The selection unit is then designed to parse input data for information about a diagnosis for a disease to determine the actual group of prediction models that should be used for selecting an individual prediction model, i.e. to filter all prediction models if they are trained with data referring to this disease.

Preferably, there is a plurality of prediction models, that are specially trained for different phases of treatment in the course of a certain disease. A preferred selection unit parses the input data regarding information about the phase of treatment and filters the prediction models accordingly (especially together with a filter regarding a certain disease) depending on their training.

As can be seen, it is preferred to label the prediction models, on what special data they are trained, e.g. could the label comprise information about a disease, a phase of treatment, patients (e.g. gender, age, BMI), medication or use cases (response to a drug or drug tapering).

Preferably, there is a plurality of prediction models, that are specially trained for different use cases (e.g. change of medication or tapering of medication). A preferred selection unit parses the input data regarding information about the use case and filters the prediction models accordingly (especially together with a filter regarding a certain disease and/or a phase of treatment) depending on their training.

Preferably, there is a plurality of prediction models, that are specially trained on lab data and other that are trained on other examination data (e.g. examination by a physician). A preferred selection unit parses the input data by looking whether there is lab/examination data available or not and filters the prediction models accordingly (especially together with a filter regarding a certain disease and/or a phase of treatment and/or a use case) depending on their training.

In an embodiment of the present invention, the patient-relating information includes information concerning distinguishing features from the group including at least one of gender, type of disease, age, underlying health condition or body mass index.

In an embodiment of the present invention, the DMARDs include bDMARDs, cDMARDs or tsDMARDs.

In an embodiment of the present invention, the clinical decision support system is configured to select the prediction model depending on whether or not lab data is part of the input data.

In an embodiment of the present invention, the number of the plurality of different trained prediction models includes at least one of: one single model to determine a drug response of a patient for a plurality of drugs, or a model of a group of multiple models to determine the drug response of a patient for a single drug.

In an embodiment of the present invention, the clinical decision support system is configured to generate, for individual parameters of the input group affecting the output the most, a value of how much the individual parameters affect the output result.

In an embodiment of the present invention, the input data is selected and provided automatically in response to new data becoming available for a patient.

In an embodiment of the present invention, at least one of: the plurality of untrained machine learning models includes a number of models having at least one of a different internal architecture or different hyperparameters, or prediction models are developed and compared offline.

In an embodiment of the present invention, for the number of the plurality of different trained prediction models, each prediction model has been trained for a different location in a clinical pathway and is selected based on input data referring to a location of the patient in a clinical pathway.

In an embodiment of the present invention, for the number of the plurality of different trained prediction models, each prediction model has been trained for a different medication and is selected based on a type of medication given in the input data, the medication being based on DMARDs or NSAIDs.

In an embodiment of the present invention, the number of the plurality of different trained prediction models are trained to determine at least one of a probability that an individual patient will respond to a specific drug or a risk of flares for different drug tapering scenarios.

Wherever not already described explicitly, individual embodiments, or their individual aspects and features, can be combined or exchanged with one another without limiting or widening the scope of the described embodiments of the present invention, whenever such a combination or exchange is meaningful and in the sense of embodiments of the present invention. Especially some features described here could form individual embodiments of the present invention, especially in combination with other features of this description. Advantages which are described with respect to one embodiment of the present invention are, wherever applicable, also advantageous of other embodiments of the present invention.

### BRIEF DESCRIPTION OF THE DRAWINGS

Other objects and features of embodiments of the present invention will become apparent from the following detailed descriptions considered in conjunction with the accompanying drawings. It is to be understood, however, that the drawings are designed solely for the purposes of illustration and not as a definition of the limits of the present invention.

FIG. 1 displays a data processing system with a CDS system according to embodiments of the present invention.

FIG. 2 displays a block diagram of a prediction-method according to embodiments of the present invention.

FIG. 3 displays a block diagram of a method for manufacturing a CDS system according to embodiments of the present invention.

FIG. 4 displays an EULAR-scheme for the treatment of rheumatoid arthritis.

FIG. 5 displays an EULAR-scheme for the treatment of psoriasis arthritis.

FIG. 6 displays a possible decision tree for the selection unit.

### DETAILED DESCRIPTION

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one example embodiment, however, may be embodied in many alternate forms and should not be construed as limited to only the example embodiments set forth herein.

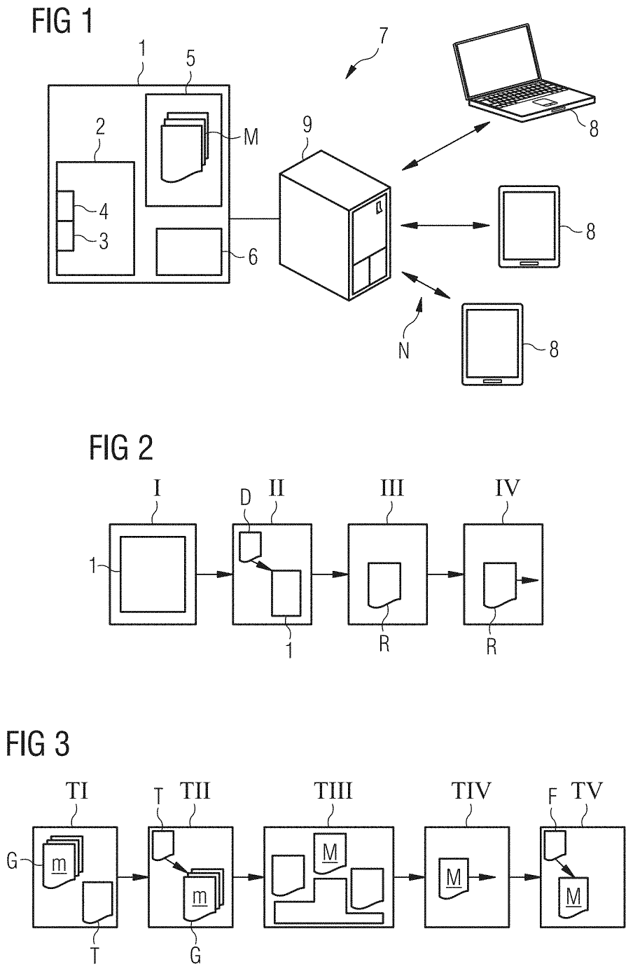

FIG. 1 displays a data processing system 7 with a CDS system 1 according to embodiments of the present invention. The data processing system 7 comprises client computers 8 connected with a service computer-system 9 via a data-network N. The service computer system 9 comprises a clinical decision support system 1 according to embodiments of the present invention.

The clinical decision support system 1 for estimating drug-related treatment optimization concerning inflammatory diseases, comprises the following components:

A computing unit 2 comprising an input interface 3 designed for receiving input data D (see following figures) and an output interface 4 designed to output results R. The computing unit 2 is designed to host a plurality of prediction models M, i.e. to process these prediction models M in order to get results R from input data D from the prediction models M. However, prediction models not used do not need to be actively hosted by the computing unit 2.

A memory 5 to save and provide the multiple prediction models M for the case a prediction model M is needed by the computing unit 2.

A plurality of different trained prediction models M that are here saved in said memory 5. Each model M is trained to predict the probability of treatment outcomes for a number of different drug-related treatment options and for a specific patient-group. For example, some prediction models M are trained for different patient-groups and should be selected based on patient-relating information in the input data D, some prediction models M are trained for different locations in a clinical pathway and are selected based on respective input data D, or some prediction models M are trained for different medications and are selected based on a type of medication given in the input data D.

The prediction models M are preferably trained to determine a probability that an individual patient will respond to a specific drug and/or a risk of flares for different drug tapering scenarios, especially for determining a response probability for first line drugs or determining a selection of the second line drug or for drug tapering in a later treatment stage (i.e. not the actual stage or phase). There could be prediction models M trained only for one single drug or for a plurality of drugs.

A selection unit 6 designed for automatically selecting one of these prediction models M depending on the input data D according to a predefined selection scheme. A prediction model M may e.g. be selected based on diagnosis, the types of input data D available, preferably wherein a prediction model M is selected depending on the case whether or not lab data is part of the input data D.

The clinical decision support system 1 is designed to produce output results R by processing the input data D with the selected prediction model M. Especially, the clinical decision support system 1 is designed to output a probability of a flare, a probability of an adverse events (e.g. side effects of medication) and/or a probability of a patient non responding to a drug. To achieve this, prediction models M of the clinical decision support system 1 may be trained to determine and output a confidence score for a prediction, preferably wherein a prediction is a binary value referring to a classification and the confidence is a probability value and/or preferably wherein the prediction is a regression the output comprises prediction intervals for point predictions.

The clinical decision support system 1 may be designed to output information (e.g. in the results) about which input group of parameters affect the output the most, preferably designed to generate for individual parameters of this group the value of how much they affect the output result R. This could e.g. be achieved by using SHAP explainable AI framework (Shapley Additive exPlanations).

FIG. 2 displays a block diagram of a prediction-method according to embodiments of the present invention.

In step I, a clinical decision support system 1 is provided, e.g. as shown in FIG. 1.

In step II, input data D is provided to the clinical decision support system 1, wherein the input data D may be selected and provided automatically, especially if new data becomes available for a predefined patient. However, also a physician can upload a chosen dataset into the CDS system 1.

In step III, a result R is determined with the clinical decision support system 1 wherein a prediction model M is selected automatically by the clinical decision support system 1 based on the input data D. The result R is determined automatically by the selected prediction model M.

In step IV, the result R is outputted by the CDS system 1. A user may be notified, if substantial changes in a result for a patient occurred compared to earlier results for the same patient, e.g. in the form of a warning message or an icon in the patient list, so that it is indicated to open the case.

FIG. 3 displays a block diagram of a method for manufacturing a CDS system 1 according to embodiments of the present invention (see e.g. FIG. 1).

It should be noted that only one single model-group G is regarded in this example, although the method uses two or more (preferably multiple) model-groups G and a respective number of training datasets T. However, the procedure is similar for each model-group G.

In step TI, a model-group G having a plurality of untrained machine learning models m is provided. It is preferred that the untrained models m have a different internal architecture and/or different hyperparameters, so it could be evaluated, which architecture/hyperparameters would be the best for a certain task.

Also in step TI, a training dataset T is provided, comprising data with a different distinguishing feature compared to other training datasets T. For example, all patients are female or the training dataset T comprises lab data.

In step TII, training of the model-group G is performed with the training-dataset T.

In step TIII, the trained prediction models M of the model-group G are ranked with predefined quality criteria. It can be seen that a trained prediction model M is the “winner” of this ranking. The prediction models M could be developed and compared offline.

In step TIV, the best ranked prediction model M of the model-group G is chosen as prediction model M for the clinical decision support system 1 manually or automatically.

In step TV, a feedback dataset F is provided for a number of patients and the (chosen) prediction models M of the CDS system 1 are further trained with this feedback dataset F. The prediction models M are here connected to the distinguishing feature of the feedback dataset F. For example, a feedback dataset F could be used for training, where a patient had a flare with a DAS28-ESR score higher than 2.6. A flare could also be self-reported by a patient, which is also included in a feedback dataset F.

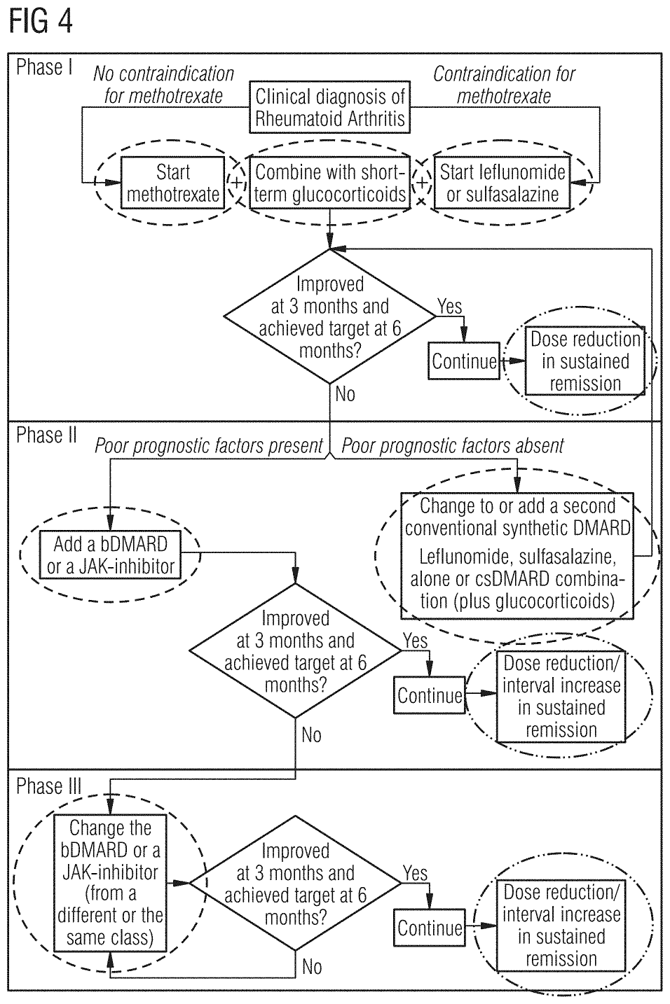

FIG. 4 displays an EULAR schematic guideline for the treatment of rheumatoid arthritis. There are three phases listed in the EULAR guidelines for treatment of RA with cDMARDs possibly in combination with glucocorticoids, bDMARDs possibly in combination with cDMARDs and tsDMARDs such as JAK-inhibitors. There are added dashed ellipses for the treatment decisions that may be supported by prediction models M specially trained for the response to drugs and dash-dotted ellipses for the treatment decisions that may be supported by prediction models M specially trained for drug tapering.

In the first phase, there is often made a selection between methotrexate and sulfasalazine combined with short term glucocorticoids. The CDS system 1 could be used to predict the effectiveness of these drugs and provide help for the estimation of the success of the applied drug. The input data D for the prediction could be demographic data and examination data of the respective patient. Predictive models M could be trained on said drugs and the response of patients concerning these drugs.

However, in this phase also a predictive model M could be selected in the case tapering of an already applied drug is planned, assuming the patient is in sustained remission. Then also input data D for the prediction could be demographic data and examination data of the respective patient together with information about the applied drug. Predictive models M could be trained on dose reduction in sustained remission scenarios of the respective drug and the response of patients to tapering.

In the second phase, more expensive active agents are typically applied. There is often made a selection between the addition of a bDMARD or a JAK-inhibitor (tsDMARD) on the one hand and the change of the already applied bDMARD or an addition of a cDMARD on the other hand. The CDS system 1 could be used to predict the effectiveness of these alternatives and provide help for the selection. The input data D for the prediction could again be demographic data and examination data of the respective patient in addition to the applied drugs. Predictive models M could be trained on said drugs and the response of patients concerning these drugs.

However, in this phase also a predictive model M could be selected in the case dose reduction or an interval increase is planned in sustained remission. Then also input data D for the prediction could be demographic data and examination data of the respective patient together with information about the applied drugs. Predictive models M could be trained on dose reduction or interval increase in sustained remission scenarios of the respective drugs and the response of patients.

In the third phase, there is often made a decision whether an applied medication should be changed (e.g. another bDMARD or a JAK-inhibitor due to poor prognostic factors or ineffectiveness or adverse events observed in the second phase). The CDS system 1 could be used to predict the effectiveness of such drug change. The input data D for the prediction could be demographic data and examination data of the respective patient in addition to the applied drugs. Predictive models M could be trained on said drugs and the response of patients concerning these drugs.

However, as in phase 2, in this phase also a predictive model M could be selected in the case dose reduction or an interval increase is planned in sustained remission. Then also input data D for the prediction could be demographic data and examination data of the respective patient together with information about the applied drugs. Predictive models M could be trained on dose reduction or interval increase in sustained remission scenarios of the respective drugs and the response of patients.

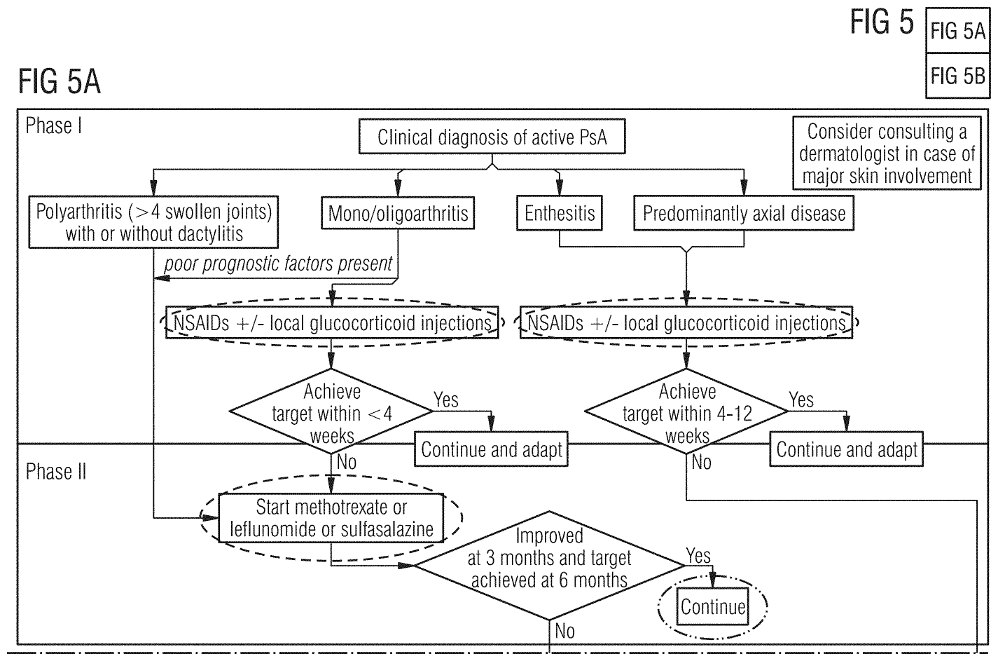

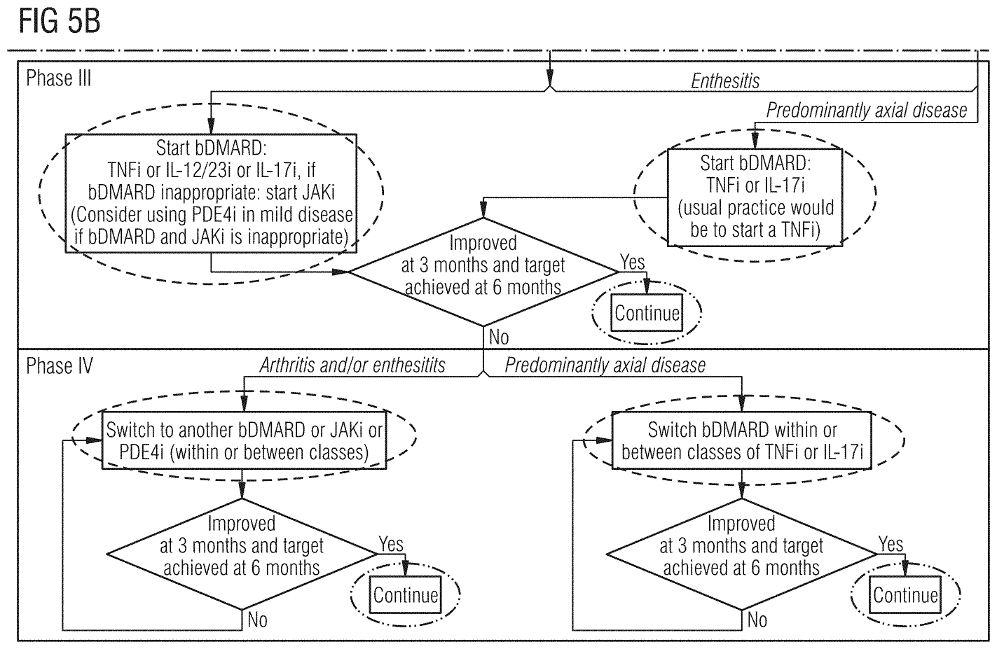

FIG. 5 displays an EULAR-scheme for the treatment of psoriasis arthritis. There are four phases listed in the EULAR guidelines for treatment of PsA, wherein the algorithm is similar to the treatment of RA. Again, there are added dashed and dash-dotted ellipses for these parts that may be predicted by predicting models M specially trained for the response to drugs.

Concerning psoriasis arthritis, there is a similar EULAR guideline comprising four phases with a similar procedure as described above. Here also, effects of the application of a new drug or the risk of flares following drug tapering could be predicted by automatically selecting a predictive model M.

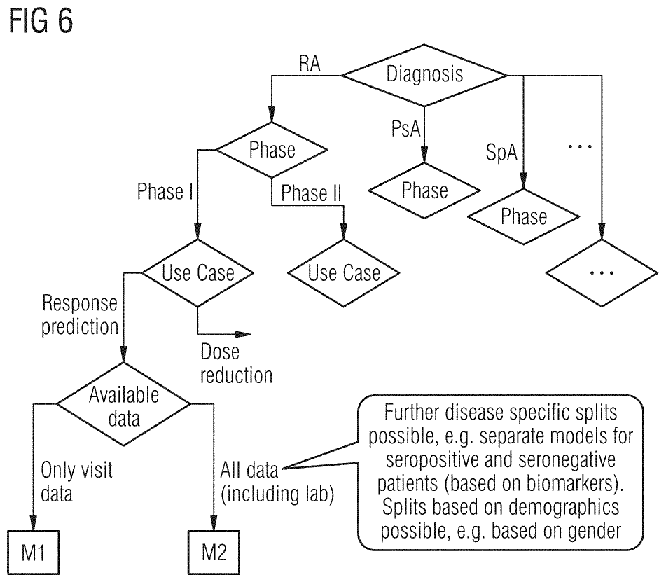

FIG. 6 displays a possible decision tree for the selection unit 6 (see above FIGS. 1 and 2).

At first (upper part), there is made a diagnosis to determine the actual disease a patient suffers from. This information is entered in the input data D and the selection unit 6 is designed to determine from the input data D the actual disease and selects prediction models M that are trained on this disease. However, there may be a vast number of possible prediction models M so that the selection should be filtered.

At second (next phase from top to bottom), the phase of treatment (see e.g. FIGS. 4 and 5) is added to the input data D and the selection unit 6 could be designed to determine from the input data D the actual phase and select prediction models M that are trained for this phase.

Third (next phase from top to bottom), the use case (change of medication or tapering of medication) could be added to the input data D and the selection unit 6 could be designed to select from the input data D prediction models M that are trained for the prediction of the influence of certain drugs on patients or the influence of drug tapering on patients.

Next (bottom phase), it could be automatically checked by the selection unit 6, whether there is examination and/or lab data available in the input data D and select prediction models M that are trained for make predictions on such data.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result.

The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Bluray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been disclosed in the form of embodiments and variations thereon, it will be understood that numerous additional modifications and variations could be made thereto without departing from the scope of the present invention.

What is claimed is:

1. A clinical decision support system for estimating drug-related treatment optimization concerning inflammatory diseases, comprising:
a computing unit configured to host a plurality of prediction models, the computing unit including an input interface configured to receive input data and an output interface configured to output results;
a plurality of different trained prediction models, wherein each model is trained to predict a probability of treatment outcomes for a number of different drug-related treatment options and for a specific patient-group based on input data; and
a selection unit configured to automatically select one of the plurality of different trained prediction models depending on the input data according to a selection scheme;
wherein the clinical decision support system is configured to produce output results by processing the input data with the selected one of the plurality of different trained prediction models.

2. The clinical decision support system according to claim 1, wherein for a number of the plurality of different trained prediction models, each prediction model has been trained for a different patient-group and is selected based on patient-relating information in the input data.

3. The clinical decision support system according to claim 1, wherein for a number of the plurality of different trained prediction models, each prediction model has been trained for a different location in a clinical pathway and is selected based on input data referring to a location of a patient in a clinical pathway.

4. The clinical decision support system according to claim 1, wherein for a number of the plurality of different trained prediction models, each prediction model has been trained for a different medication and is selected based on a type of medication given in the input data, the medication being based on DMARDs or NSAIDs.

5. The clinical decision support system according to claim 1, wherein the clinical decision support system is configured to select a prediction model based on types of input data available.

6. The clinical decision support system according to claim 1, wherein a number of the plurality of different trained prediction models are trained to determine at least one of a probability that an individual patient will respond to a specific drug or a risk of flares for different drug tapering scenarios.

7. The clinical decision support system according to claim 1, wherein a number of the plurality of different trained prediction models are trained to determine drug response of a patient for a plurality of drugs.

8. The clinical decision support system according to claim 1, wherein the clinical decision support system is configured to output at least one of a probability of a flare, a probability of an adverse event or a probability of a patient not responding to a drug.

9. The clinical decision support system according to claim 1, wherein the clinical decision support system is configured to output information about which input group of parameters affect the output the most.

10. A method comprising:
providing a clinical decision support system according to claim 1;
providing input data to the clinical decision support system, wherein the input data is selected and provided automatically;
determining a result with the clinical decision support system, wherein a prediction model is selected automatically by the clinical decision support system based on the input data and the result is determined automatically by the selected prediction model; and
outputting the result.

11. A method for manufacturing a clinical decision support system according to claim 1, the method comprising:
providing at least a first model-group and a second model-group, each model-group having a plurality of untrained machine learning models;
providing at least a first training-dataset and a second training-dataset, each training-dataset including data with a different distinguishing feature;
training the first model-group with the first training-dataset and the second model-group with the second training-dataset;
ranking each trained prediction model of a model-group with quality-criteria; and
choosing the best ranked prediction model of each model-group as prediction model for the clinical decision support system.

12. The method according to claim 11, wherein a prediction method is performed with the clinical decision support system and a feedback-dataset is provided for a number of patients, wherein the trained prediction models are further trained with this feedback dataset, the trained prediction models being connected to the distinguishing feature of the feedback data, wherein a feedback-dataset in which a patient had a flare with a DAS28-ESR score higher than 2.6 is used for training.

13. A data processing system, comprising:
a data-network,
a number of client computers, and
a service computer system, the service computer system including the clinical decision support system according to claim 1.

14. A non-transitory computer program product comprising a computer program that is directly loadable into a memory of a control unit of a computer system and which comprises program elements that, when executed at the control unit, cause the control unit to perform the method according to claim 10.

15. A non-transitory computer-readable medium storing program elements that, when executed by a computer unit, cause the computer unit to perform the method according to claim 10.

16. The clinical decision support system according to claim 2, wherein the patient-relating information includes at least one of demographic data or examination data.

17. The clinical decision support system according to claim 3, wherein the input data is examination data.

18. The clinical decision support system according to claim 6, wherein a prediction model is trained for at least one of
determining a response probability for a first line drug,
determining a selection of a second line drug,
a drug tapering scenario in a later treatment stage for RA patients receiving biologics in stable remission, or
a plurality of dosage regimes.

19. The clinical decision support system according to claim 8, wherein
the clinical decision support system is configured to output the probability of the flare connected to at least one of an application or a dosage of a medication, and
at least one of
the plurality of different trained prediction models of the clinical decision support system are trained to determine and output a confidence score for a prediction,
the prediction is a binary value referring to a classification,
the confidence score is a probability value,
the prediction is a regression, or
the output includes prediction intervals for point predictions.

20. The method of claim 10, wherein the outputting comprises:
notifying a user in response to changes in a result for a patient compared to earlier results for the patient, wherein
the notifying notifies the user in the form of a warning message or an icon in a patient list.
